(19) United States
(12) Patent Application Publication
(10) Pub. No.: US 2018/0273909 A1
(43) Pub. Date: Sep. 27, 2018

(54) PURIFICATION OF RESPIRATORY SYNCYTIAL VIRUS

(71) Applicant: Sanofi Pasteur Biologics, LLC, Cambridge, MA (US)

(72) Inventor: Sophia Tara MUNDLE, Boston, MA (US)

(21) Appl. No.: 15/761,913

(22) PCT Filed: Sep. 19, 2016
(86) PCT No.: PCT/US2016/052515

§ 371 Date: Mar. 21, 2018

Related U.S. Application Data
(60) Provisional application No. 62/221,874, filed on Sep. 22, 2015.

(51) Int. Cl. C12N 7/00; A61K 35/76; A61K 47/26; B01D 63/02; B01D 15/34; B01D 15/38; B01D 61/14; B01D 71/68

(52) U.S. Cl. CPC C12N 7/00; A61K 35/76; A61K 47/26; B01D 63/02; B01D 15/34; C12N 2760/00064; B01D 61/145; B01D 71/68; C12N 2760/00021; C12N 2760/00051; B01D 15/3847

(57) ABSTRACT

Disclosed herein are methods for the purification of Respiratory Syncytial Virus (RSV) particles from a host cell culture comprising treating the host cell culture with an endonuclease, filtering the material to remove cellular debris and/or aggregated material, applying the material to a core bead chromatography resin, and recovering the purified RSV particles. Also disclosed herein are pharmaceutical compositions comprising purified RSV.

### CROSS REFERENCE TO RELATED APPLICATIONS

This application claims the benefit of, and relies on the filing date of, U.S. provisional patent application No. 62/221,874, filed 22 Sep. 2015, the entire disclosure of which is herein incorporated by reference.

### FIELD OF THE DISCLOSURE

Disclosed herein are methods for purifying viral particles and compositions comprising the same.

### BACKGROUND OF THE DISCLOSURE

Respiratory Syncytial Virus (RSV) is an important human pathogen, causing disease in children and frequently causing severe lower respiratory tract infections in infants, as well as the elderly and immunocompromised. Although a passive prophylactic treatment does exist for susceptible neonates and children, its safety and efficacy have not been demonstrated for treatment of established RSV disease. Thus the overall disease burden warrants the development of a safe and effective active prophylactic vaccine for use in otherwise healthy newborns and children. It is estimated that human respiratory syncytial virus (hRSV) causes up to 200,000 deaths per year worldwide in children younger than 5 years of age (Nair, H. et al., Lancet 2010; 375:1545-55). Furthermore, approximately 33 million children of the same age group suffer from acute lower respiratory infection due to hRSV, with at least 10% of those cases being severe enough to require hospitalization.

Therapeutic intervention in humans is limited to treatment with the antiviral molecule Rivabirin. Use of this molecule, however, is uncommon and controversial due to the potential for side effects and concern about cost and efficacy (Law, B. et al., Pediatrics, 1997; 99:E7; Ventre, K. et al., Cochrane Database Syst Rev., 2007: CD000181). To date the safe and effective induction of protective immunity in humans has thus not been accomplished by active vaccination, although extensive testing of candidates has been undertaken both in animal models and in humans.

In addition to peptide-based vaccine approaches, the live attenuated virus (LAV) vaccine approach has been explored (Friedewald, W. et al., JAMA, 1968; 204:690-4; Karron R., et al., J Infect Dis., 2005; 191:1093-104; Luongo C, et al., J Virol., 2013; 87:1985-96; Schickli J. et al., Virus Res., 2012; 169:38-47; Wright P., et al., J Infect Dis., 2000; 182:1331-42). Temperature sensitive, gene deletion, and passage-attenuated approaches have all been tested in animals and, in some cases, in humans. One of the challenges to bringing a LAV vaccine candidate to the clinic, however, is the high level of purity that is required for injection into humans. Since immunogenicity of these candidates is dependent on the ability to infect cells, preservation of infectious titer is important to processes designed for purification of LAVs, and RSV is known to be an extremely labile virus, prone to aggregation and loss of infectivity during handling and preparation, including bind and elute chromatography.

Chromatographic separation procedures have previously been described for preparation of difficult to work with enveloped viruses such as herpes simplex virus type 2 (HSV2) and Flaviviruses. See, e.g., Mundle et al., PLoS One, 2013; 8:e57224 (discussing HSV2). Purification of hRSV has traditionally been performed by ultracentrifugation in either sucrose or iodixanol, with recoveries of infectious virus up to about 60-70% (Liljeroos, L, et al., Proc Natl Acad Sci USA, 2013; 110:11133-8; Radhakrishnan, A, et al., Mol Cell Proteomics, 2010; 9:1829-48; Mbiguino, A., et al., J Virol Methods, 1991; 31:161-70; Ueba, O., et al., Acta Med Okayama, 1978; 32:265-72; Gias, E., J Virol Methods, 2008; 147:328-32). Additionally, chromatographic purification of viral proteins from cell culture-derived RSV has been described (Ling, Z. et al., Protein Expr Purif, 2008; 57:261-70; Zheng Y., et al., Protein Expr Purif, 2012; 81:115-8). Although there is a report of an ion exchange chromatography-based purification scheme for RSV, recovery of infectious virus in that case was only about 1% (Downing, L. A., et al., J Virol Methods, 1992; 38:215-28).

These traditional laboratory-scale purification processes for vaccine strain viruses involve laborious procedures that cannot be scaled for commercial production of viral compositions prepared in accordance with World Health Organization (WHO) guidelines for human use, resulting in either low yields or insufficient purity (e.g., excessively high levels of residual host cell DNA). The WHO provides an upper limit of 10 ng host cell DNA per human dose. Therefore, a need exists to provide virus preparations with less than 10 ng host cell DNA per human dose.

Disclosed herein are scalable, chromatography-based purification procedures for the preparation of highly pure, infectious RSV that may be of similar potency to crude, unpurified material when tested in vivo. The purification schemes disclosed herein are based on core bead technology and hollow fiber tangential flow filtration (TFF) and, in certain embodiments, may result in at least about 60% recovery of infectious virus titer. The methods disclosed herein can be used to prepare highly purified wild type or live-attenuated vaccine strain viruses with titers of greater than about 1×10⁸ plaque forming units per mL. RSV prepared by this method may be about 50 to about 200-fold more pure with respect to dsDNA and host cell proteins, as compared to the raw feed stream. The methods disclosed herein can be considered a starting point for downstream process development of a live-attenuated vaccine approach for prevention of infection by RSV.

### SUMMARY OF THE DISCLOSURE

The present disclosure provides methods to prepare purified RSV employing core bead flowthrough chromatography and tangential flow filtration, such as hollow fiber tangential flow filtration. These methods can be used to prepare high yield viral preparations, including RSV preparations, in accordance with WHO guidelines for human use, including high purity (e.g., less than 10 ng host cell DNA per exemplary human dose (e.g., 1×10⁷ PFU or 1×10⁸ PFU)).

One aspect of this disclosure is directed to a method for the purification of RSV particles from a mammalian host cell culture comprising the steps of:
a) treating the mammalian host cell culture with an endonuclease;
b) filtering the material from step (a) to remove cellular debris and/or aggregated material;
c) applying the material obtained from step (b) to a core bead chromatography resin such that the RSV particles flow through the core bead chromatography resin;
   and
d) recovering the purified RSV particles.

In various embodiments of the disclosure, the method further comprises subjecting the RSV particles recovered in step (d) to tangential flow filtration. In some embodiments, the purified RSV particles contain greater than about 1×10⁷ or about 2×10⁷ plaque forming units (PFU)/mL, such as greater than about 1×10⁸ PFU/mL. In other embodiments, the purified RSV particles contain less than 10 ng host cell DNA per 1×10⁷ plaque forming units (PFU) or less than 10 ng host cell DNA per 1×10⁸ PFU. In certain embodiments, the RSV is a live attenuated virus (LAV) strain, and in some embodiments the RSV is a wild type virus strain.

In various embodiments disclosed herein, the endonuclease is an endonuclease from Serratia marcescens and comprises two subunits, each of which has a molecular weight of about 30 kD, and degrades double stranded and single stranded DNA and double stranded and single stranded RNA and is sold under the trademark Benzonase®. According to certain embodiments, in the methods disclosed herein, greater than about 90%, such as greater than about 95% or greater than about 99%, of the host cell protein is removed in the recovered purified RSV particles. In certain embodiments, greater than about 90%, such as greater than about 95%, of the host cell DNA is removed in the recovered purified RSV particles. In certain embodiments of the disclosure, about 100% of the infectious RSV titer from the host cell culture remains following the core bead chromatography step, and in certain embodiments, about 50-60% of the infectious RSV titer from the host cell culture remains following the tangential flow filtration step.

One aspect of the disclosure is directed to a method for the purification of RSV particles from a mammalian host cell culture comprising the steps of:
a) treating the mammalian host cell culture with an endonuclease;
b) filtering the material from step (a) to remove cellular debris and/or aggregated material;
c) applying the treated mammalian host cell culture to a core bead chromatography resin such that the RSV particles flow through the core bead chromatography resin;
d) collecting the RSV particles;
e) subjecting the RSV particles collected in step (d) to tangential flow filtration;
   and
f) recovering the purified RSV particles.

In certain embodiments disclosed herein, the tangential flow filtration is a hollow fiber system. In one embodiment, the hollow fiber system has a molecular weight cutoff of 100 kDa. In another embodiment, the chromatography system comprises a core bead chromatography resin, such as the Capto™ Core 700 by GE Healthcare Life Sciences.

According to various embodiments disclosed herein wherein the RSV particles are subjected to a tangential flow filtration step, about 50-60% of the infectious RSV titer from the host cell culture may remain following the tangential flow filtration step. In some embodiments, the purified RSV particles contain greater than about 1×10⁷ or about 2×10⁷ plaque forming units (PFU)/mL, such as greater than about 1×10⁸ PFU/mL. In other embodiments, the purified RSV particles contain less than 10 ng host cell DNA per 1×10⁷ plaque forming units (PFU) or less than 10 ng host cell DNA per 1×10⁸ PFU.

In various embodiments disclosed herein wherein the RSV particles are subjected to a tangential flow filtration step, the endonuclease is Benzonase®. In other methods disclosed herein, greater than about 90%, such as greater than about 95% or greater than about 99%, of the host cell protein is removed in the recovered purified RSV particles. In certain embodiments, greater than about 90%, such as greater than about 95%, of the host cell DNA is removed in the recovered purified RSV particles.

Another aspect of the disclosure is directed to a pharmaceutical composition comprising RSV produced in a mammalian cell culture, said RSV isolated by the method comprising the steps of:

a) treating the mammalian host cell culture with an endonuclease;

b) filtering the material from step (a) to remove cellular debris and/or aggregated material;

c) applying the material obtained from step (b) to a core bead chromatography resin such that the RSV particles flow through the core bead chromatography resin;

d) recovering the purified RSV particles; and

e) suspending the purified RSV particles in a pharmaceutically acceptable carrier.

In various embodiments of the pharmaceutical compositions disclosed herein, the method further comprises subjecting the RSV particles recovered in step (d) to tangential flow filtration. In yet another embodiment, the quantity of host cell DNA in said composition is less than 10 ng host cell DNA per 1×10⁷ or 1×10⁸ plaque forming units (PFU). In certain embodiments, the RSV is a LAV strain, and in certain embodiments, the RSV is a wild type virus strain. In other embodiments of the disclosure, the composition contains greater than about 1×10⁷ PFU/mL, such as greater than about 2×10⁷ PFU/mL, or about 1×10⁸ PFU/mL.

In various embodiments disclosed herein, the endonuclease is an endonuclease from Serratia marcescens and comprises two subunits, each of which has a molecular weight of about 30 kD, and degrades double stranded and single stranded DNA and double stranded and single stranded RNA and is sold under the trademark Benzonase®. According to certain embodiments, in the pharmaceutical composition disclosed herein, greater than about 90%, such as greater than about 95% or greater than about 99%, of the host cell protein is removed in the recovered purified RSV particles. In certain embodiments, in the pharmaceutical composition disclosed herein, greater than about 90%, such as greater than about 95%, of the host cell DNA is removed in the recovered purified RSV particles. In certain embodiments of the pharmaceutical compositions disclosed herein, about 100% of the infectious RSV titer from the host cell culture remains following the core bead chromatography step, and in certain embodiments, about 50-60% of the infectious RSV titer from the host cell culture remains following the tangential flow filtration step.

Another aspect of the disclosure is directed to a composition comprising RSV particles in a buffer comprising sorbitol, such as a buffer comprising potassium glutamate, L-histidine, sodium chloride, and sorbitol.

Another aspect of the disclosure is directed to a pharmaceutical composition comprising Respiratory Syncytial Virus (RSV) produced in a cell culture, said RSV isolated by the method comprising the steps of:
a) treating the host cell culture with an endonuclease;
b) filtering the material from step (a) to remove cellular debris and/or aggregated material;
c) applying the material obtained from step (b) to a core bead chromatography resin such that the RSV particles flow through the resin;
d) collecting the RSV particles;
e) subjecting the RSV particles collected in step (d) to tangential flow filtration;
f) recovering the purified RSV particles; and
g) suspending the purified RSV particles in a pharmaceutically acceptable carrier.

In various embodiments disclosed herein wherein the RSV particles of the pharmaceutical composition are subjected to a tangential flow filtration step, the tangential flow filtration is a hollow fiber system. In certain embodiments, the quantity of host cell DNA in said composition is less than 10 ng host cell DNA per 1×10′ plaque forming units (PFU) or less than 10 ng host cell DNA per 1×10⁸ PFU. In other embodiments of the disclosure wherein the RSV particles of the pharmaceutical composition are subjected to a tangential flow filtration step, the composition contains greater than about 1×10⁷ PFU/mL, such as greater than about 2×10⁷ PFU/mL, or about 1×10⁸ PFU/mL.

In various embodiments of the pharmaceutical composition disclosed herein wherein the RSV particles are subjected to a tangential flow filtration step, the endonuclease is from Serratia marcescens and comprises two subunits, each of which has a molecular weight of about 30 kD, and degrades double stranded and single stranded DNA and double stranded and single stranded RNA and is sold under the trademark Benzonase®. According to certain embodiments, disclosed herein, greater than about 90%, such as greater than about 95% or greater than about 99%, of the host cell protein is removed in the recovered purified RSV particles. In certain embodiments of the pharmaceutical composition disclosed herein wherein the RSV particles are subjected to a tangential flow filtration step, greater than about 90%, such as greater than about 95%, of the host cell DNA is removed in the recovered purified RSV particles.

Another aspect of the disclosure is directed to a composition comprising RSV particles, wherein the RSV particles have been subjected to a tangential flow filtration step, in a buffer comprising sorbitol, such as a buffer comprising potassium glutamate, L-histidine, sodium chloride, and sorbitol.

### BRIEF DESCRIPTION OF THE FIGURES

FIG. 1 is a representative chromatographic profile during laboratory scale purification of RSV by core bead chromatography. The solid line represents absorbance at 280 nm. The dotted line represents the concentration of Buffer B, a Cleaning-In-Place (CIP) solution of 0.5M NaOH in 30% isopropyl alcohol, which followed the sample flowthrough phase to remove bound impurities.

FIG. 2 shows a comparison of purified live-attenuated RSV particles prepared by core bead chromatography and TFF. SDS-PAGE with Coomassie Brilliant Blue staining (CBB) and western blot (α-RSV-F, α-RSV-G, and α-RSV-M2-1) analysis revealed both enrichment and concentration of viral proteins. The lanes represent the various purification fractions: (1) unpurified; (2) benzonase-treated; (3) 0.65 μm depth-filtered; (4) Capto™ Core 700 flowthrough fraction; (5) Capto™ Core 700 CIP; (6) TFF permeate 1; (7) TFF permeate 2; and (8) TFF retentate or purified RSV.

FIG. 3A is a transmission electron micrograph of partially-purified, live-attenuated RSV at a scale of 500 nm.

FIG. 3B is a transmission electron micrograph of partially-purified, live-attenuated RSV at a scale of 100 nm.

FIG. 3C is a transmission electron micrograph of partially-purified, live-attenuated RSV at a scale of 20 nm, showing clearly visible glycoprotein spikes at the surface of the particles.

FIG. 4 is a graph illustrating the preparation of whole cell lysate by mechanical cell disruption, showing that sonication and low pressure microfluidization resulted in 2-fold higher titers as compared to the amount of infectious virus in the clarified cell culture supernatant.

FIG. 5A shows a small-scale comparison of initial purification steps using supernatant as the bulk harvest material. From left-to-right are chromatograms, SDS-PAGE and western blots (α-RSV-F and α-RSV-G). The lanes represent the various purification fractions: MW marker is in lane 1, and RSV-containing samples are in the other lanes, from left-to-right in the following order: unpurified, Benzonase®-treated, 0.8 μm filtered, Capto™ Core 700 flowthrough fraction, and Capto™ Core 700 CIP.

FIG. 5B shows a small-scale comparison of initial purification steps using whole cell lysate prepared by sonication as the bulk harvest material. From left-to-right are chromatograms, SDS-PAGE and western blots (α-RSV-F and α-RSV-G). The lanes represent the various purification fractions: MW marker is in lane 2, and RSV-containing samples are in the other lanes, from left-to-right in the following order: unpurified, Benzonase®-treated, 0.8 μm filtered, Capto™ Core 700 FT fraction, and Capto™ Core 700 CIP.

FIG. 5C shows a small-scale comparison of initial purification steps using whole cell lysate prepared by microfluidization as the bulk harvest material. From left-to-right are chromatograms, SDS-PAGE and western blots (α-RSV-F and α-RSV-G). The lanes represent the various purification fractions: MW marker is in lane 3, and RSV-containing samples are in the other lanes, from left-to-right in the following order: unpurified, Benzonase®-treated, 0.8 nm filtered, Capto™ Core 700 FT fraction, and Capto™ Core 700 CIP.

FIG. 6A is a study timeline for the immunogenicity and protective efficacy of a LAV strain in cotton rats.

FIG. 6B is a graph showing neutralizing antibody titers in cotton rat serum collected on day 28 post-immunization with 1×10⁴ PFU intramuscularly. The dotted line represents the limit of detection.

FIG. 6C is a graph showing lung and nasal RSV titers, 4 days post-challenge, from cotton rats challenged with 1×10⁵ PFU of long strain RSV intranasally on day 28 post-immunization. The dotted line represents the limit of detection.

### DETAILED DESCRIPTION OF THE DISCLOSURE

In order to advance development of RSV vaccines into animal models and clinical studies, disclosed herein are scalable processes capable of producing viral material suitable for mammalian use. Highly-purified RSV may be made by processing of RSV-infected host cells, such as Vero cells, by endonuclease treatment, depth filtration, core bead flowthrough chromatography, and optionally tangential flow filtration. In certain embodiments, the purification schemes disclosed herein yield virus that is sufficiently pure with respect to residual host cell genomic DNA for testing in humans (such as, for example, less than 10 ng residual host cell DNA per 1×10⁷ PFU). The terms “virus” and “virus particles” are used interchangeably herein.

In one aspect, the present disclosure provides a method for the purification of an enveloped viral particle, such as an RSV particle, from a mammalian host cell culture comprising the steps of:
a) treating the mammalian host cell culture with an endonuclease;
b) applying the treated mammalian host cell culture to a core bead chromatography resin such that the RSV particles flow through the core bead chromatography resin; and
c) collecting the purified RSV particles.

In certain embodiments, the method may further comprise a tangential flow filtration step. For example, in certain embodiments the tangential flow filtration may be a hollow fiber tangential flow filtration. In embodiments comprising a tangential flow filtration step, the tangential flow filtration step may occur before or after the material is applied to a core bead chromatography resin.

Accordingly, the present disclosure further provides a method for the purification of an enveloped viral particle, such as RSV, from a mammalian host cell culture comprising the steps of:
a) treating the mammalian host cell culture with an endonuclease;
b) applying the treated mammalian host cell culture to a core bead chromatography resin such that the RSV particles flow through the core bead chromatography resin;
c) collecting the RSV particles;
d) subjecting the RSV particles collected in step (c) to tangential flow filtration;
   and
e) recovering the purified RSV particles.

In certain exemplary embodiments, the method may further comprise clarifying the material with a depth filtration step in order to remove any cellular debris and/or aggregated material. In certain embodiments, the depth filtration step may be carried out prior to the core bead chromatography step.

Accordingly, the present disclosure further provides a method for the purification of an enveloped viral particle, such as an RSV particle, from a mammalian host cell culture comprising the steps of:
a) treating the mammalian host cell culture with an endonuclease;
b) filtering the material from step (a) to remove cellular debris and/or aggregated material;
c) applying the material obtained from step (b) to a core bead chromatography resin such that the RSV particles flow through the core bead chromatography resin;
d) collecting the RSV particles;
e) subjecting the RSV particles collected in step (d) to tangential flow filtration;
   and
f) recovering the purified RSV particles.

Also disclosed herein are methods for the purification of an enveloped viral particle, such as an RSV particle, from a mammalian host cell culture comprising the steps of:
a) treating the mammalian host cell culture with an endonuclease;
b) filtering the material from step (a) to remove cellular debris and/or aggregated material;

c) applying the material obtained from step (b) to a core bead chromatography resin such that the RSV particles flow through the core bead chromatography resin; and

d) collecting the purified RSV particles.

Typical mammalian cell hosts for enveloped viruses are well known to those of skill in the art and are readily available from public and private depositories. Particularly useful for the production of viruses disclosed herein here for purposes of the present disclosure include the Vero, HEK293, MDK, A549, EB66, CHO and PERC.6 host cells.

RSV is one of the negative-sense, single-stranded RNA viruses. It belongs to the family Paramyxoviridae, and is a member of the genus Pneumovirus. Pneumoviruses include pathogens that work specifically to target the respiratory tract and may result in serious infections such as bronchiolitis or pneumonia.

There is a range of time after infection of the host cells with RSV where the maximum amount of virus can be released from the cells. The timing of release may vary depending on the temperature, the infection media used, the virus that was used to infect the cells, the container in which the cells were grown and infected, and the cells themselves. Identification of this optimal harvest time may be determined by sampling of the cell culture regularly over the conventional incubation period for the particular enveloped virus to determine the optimal yield. Under the conditions reported in the Examples below (Vero cells; LAV and wild type MSA-1), the virus-containing media was harvested at 6 days post infection.

In embodiments disclosed herein, the viral-containing cell culture may be harvested by any method known in the art, including, for example collection of the supernatant after centrifugation or collection of whole cell lysate after mechanical cell disruption. For example, the cell culture may be subjected to sonication, or, in certain embodiments, microfluidization, such as low pressure microfluidization, in order to prepare a whole cell lysate.

The disclosure further provides methods as described herein wherein the endonuclease used to degrade residual host cell DNA is Benzonase®. In certain embodiments, the endonuclease may be one that degrades both DNA and RNA. In one embodiment, for example, the endonuclease is a genetically engineered endonuclease from Serratia marcescens (Eaves, G. N. et al. J. Bact. 1963, 85, 273-278; Nestle, M. et al. J. Biol. Chem. 1969, 244, 5219-5225; U.S. Pat. No. 5,173,418, which is hereby incorporated by reference in its entirety) that is sold under the name Benzonase® (EMD Millipore). The Benzonase® endonuclease from Serratia marcescens comprises two subunits, each with a molecular weight of about 30 kD and degrades all forms of DNA and RNA (single stranded, double stranded, linear and circular) and may be effective over a wide range of operating conditions, digesting nucleic acids to 5′-monophosphate terminated oligonucleotides 2 to 5 bases in length in the presence of divalent metal cations, such as Mg²⁺. Benzonase® has an isolectric point at pH 6.85. Benzonase® is produced under current good manufacturing practices (cGMP) and, thus, can be used in industrial scale processes for the purification of proteins and/or viral particles. Other endonucleases that are produced under cGMP conditions can likewise be used in the purification methods disclosed herein.

In certain embodiments of the purification methods disclosed herein, following the endonuclease step, the material may be filtered, for example by depth filtration. Depth filtration may be used to remove cellular debris and/or aggregated material, such as, for example, host cell proteins and host cell DNA. As understood in the art, depth filtration refers to the use of a porous filter medium to clarify solutions containing significant quantities of large particles (e.g., intact cells or cellular debris) in comparison to membrane filtration, which may rapidly become clogged under such conditions. A variety of depth filtration media of varying pore sizes are commercially available from a variety of manufacturers such as Millipore, Pall, General Electric, and Sartorious. In the practice of the disclosure as exemplified herein, SartoScale disposable Sartopure PP2, 0.65 μm depth filters (Sartorious Stedim, Goettingen, Germany) were used.

When performing a depth filtration procedure prior to a core bead chromatography, endonuclease treatment of the viral preparation prior to depth filtration may improve the efficiency of the process by minimizing fouling of the depth filtration matrix. Alternatively, even in the absence of a depth filtration step, the recovery of virus from the chromatographic step may be diminished when non-endonuclease treated virus is applied to this and other chromatographic supports.

According to embodiments disclosed herein, following endonuclease treatment and depth filtration, the viral material to be purified may be subjected to a core bead chromatography resin. Bind-and-elute chromatography resins, including a low-shear anion exchange membrane (Mustang Q) and monolith (convective interaction media (CIM) Q) and affinity matrices (Cellufine Sulfate, Capto DeVirS and HiTrap Heparin HP), were tested at the small scale but all resulted in poor recovery of infectious material post-elution. Recoveries ranged between 0 and 40%.

In core bead chromatography, molecules may be separated based on size. Larger molecules flow through the chromatography column, while smaller molecules flow into pores on the surface of the bead. The pore size on the surface of the bead will determine the size of the molecule that may pass through to the inner core of the bead. Accordingly, one skilled in the art may select a core bead resin with an appropriate pore size smaller than the molecule (such as, for example, the RSV virus) that is the subject of purification, such that the molecule to be purified passes through the column, while smaller molecules enter the pores of the core bead resin. In certain exemplary embodiments, the core bead resin comprise pores having an approximate molecular weight cutoff (MWCO) of about 700 kDa. In certain embodiments disclosed herein, the inner core of the bead may comprise functionalized ligands that act to bind the particles that flow through to the inner core, such as, for example, impurities and/or host cell proteins.

Recently, core bead technology has been released as part of GE Healthcare Life Sciences BioProcess™ line of chromatography resins. Specifically, Capto™ Core 700 is a resin that combines size separation and binding chemistry in a single chromatographic matrix, which may result in improved process productivity for the production of large molecules such as viruses. Indeed, a process has been recently described for production of influenza virus from allantoic fluid, which rivals the purity (as measured by ovalbumin removal) achieved using more common methods such as zonal ultracentrifugation (Blom, H. et al., Vaccine, 2014; 32:3721-4).

Capto™ Core 700 is a layered, bead-based matrix having a particle size of about 90 μm. The surface of the bead consists of an unliganded, inactive shell with pores that have an approximate MWCO of about 700 kDa. The interior of the bead comprises an active functionalized core with multimodal octylamine ligands designed to capture impurities that are small enough to enter the bead through the pores on the surface. Smaller molecules, such as impurities and/or host cell proteins having a size smaller than about 700 kDa, pass through to the inner core octylamine ligands, where they are adsorbed, while the larger virion particles flow through. Large molecules can thereby be purified from smaller contaminants in the negative (flowthrough) purification mode. Capto™ Core 700 is therefore a flowthrough chromatography resin that may be used to purify viruses and/or other large biomolecules.

As the octaylamine ligands of the inner core are both hydrophobic and positively charged, they allow various impurities to efficiently bind thereto over a wide range of pH and salt concentrations. The bound impurities may be removed from the beads by a process known as cleaning-in-place (CIP), wherein a solution is passed through the beads to elute the bound impurities. In certain embodiments disclosed herein, sodium hydroxide and optionally a solvent, such as, for example, 1M NaOH in 27% 1-propanol or 0.5M NaOH in 30% isopropyl alcohol, may be used as a CIP solution.

In certain embodiments, the methods disclosed herein may further comprise subjecting the material to tangential flow filtration (TFF), either prior to or after passing the material through the core bead chromatography resin. In certain exemplary embodiments, the methods disclosed herein further comprise subjecting the material to TFF after the core bead chromatography step. TFF, also referred to as Cross Flow Filtration (CFF), is well known to those of skill in the art, and equipment and protocols for its implementation in a wide range of situations are commercially available from a variety of manufacturers, including but not limited to the Pall Corporation, Port Washington, N.Y. and Spectrum Labs, Rancho Dominguez, Calif. TFF can be used to concentrate and/or exchange buffers in sample solutions ranging in volume from 10 mL to thousands of liters. It can also be used to fractionate large from small biomolecules, harvest cell suspensions, and clarify fermentation broths and cell lysates.

Generally, TFF involves the recirculation of the retentate across the surface of the membrane. This gentle cross flow feed minimizes membrane fouling, maintains a high filtration rate, and provides high product recovery. In one embodiment, the TFF step may be implemented with a flat sheet system. Flat sheet systems may be used in large scale production where such systems are provided with a means (e.g., an open flow channel) to prevent excessive shear forces on the enveloped viral particles. Alternatively, the TFF step may be implemented with a hollow fiber system, as exemplified herein. In one embodiment, the MWCO of the TFF system ranges from about 50 kDa to about 1000 kDa, such as from about 50 kDa to about 250 kDa or from about 250 kDa to about 500 kDa. In certain embodiments, the MWCO of the TFF system is about 100 kDa, about 200 kDa, or about 500 kDa.

In certain embodiments, the viral (e.g., RSV) particles purified according to the methods disclosed herein may be produced in high yield and with sufficient purity that they can be administered to a humans. For example, according to certain exemplary embodiments, the methods disclosed herein may produce purified RSV particles comprising less than 10 ng residual host cell DNA per 1×10⁷ plaque forming units (PFU).

In some embodiments, the purified viral (e.g., RSV) particles may contain greater than about 1×10⁷ PFU/mL, such as greater than about 2×10⁷ PFU/mL or greater than about 1×10⁸ PFU/mL.

In certain embodiments, purified RSV particles may be obtained in yields greater than about 80%, such as greater than about 90%, or about 100% of the infectious titer of virus in the solution obtained by subjecting the host cell culture to core bead chromatography.

In other exemplary embodiments, the purified RSV particles may contain greater than about 90%, such as greater than about 95%, of the infectious titer of virus in the solution obtained by treating the host cell culture with an endonuclease, such as Benzonase®. In certain embodiments, the purified RSV particles may contain greater than about 80%, such as greater than about 85%, greater than about 90%, greater than about 95%, or about 100% of the infectious titer of virus in the solution obtained by subjecting the host cell culture to depth filtration after treating the host cell culture with an endonuclease.

In certain embodiments disclosed herein, the purified RSV particles may contain greater than about 90%, such as greater than about 95%, or about 100% of the infectious titer of virus in the solution obtained by subjecting the host cell culture to core bead chromatography after treating the host cell culture with an endonuclease, such as, for example, Benzonase®. In certain embodiments disclosed herein, the purified RSV particles may contain a range of about 85% to about 100%, such as about 90% to about 100%, about 95% to about 100%, or about 96% to about 100%, of the infectious titer of virus in the solution obtained by subjecting the host cell culture to core bead chromatography after treating the host cell culture with an endonuclease, such as, for example, Benzonase®. RSV, like many enveloped viruses, is pleomorphic and extremely fragile, which may make it more difficult to purify. Traditional chromatography methods may subject the viral particles to excessive forces such as shear, resulting in a subsequent loss of yield and infectivity. Previous attempts at purification of RSV through ion exchange chromatography, for example, resulted in recovery of infectious virus of only about 1% (Downing, L. A., et al., J Virol Methods, 1992; 38:215-28). Therefore, the present disclosure of obtaining purified RSV particles in yields greater than about 90%, such as greater than about 95%, or about 100% of the infectious titer of virus in the solution obtained by subjecting the host cell culture to core bead chromatography is surprising and unexpected.

In certain embodiments, the purified RSV particles may contain greater than about 50%, such as about 60%, about 65%, or greater than about 65%, of the infectious titer of virus in the solution obtained by subjecting the host cell culture to TFF after subjecting the host cell culture to core bead chromatography. In certain embodiments disclosed herein, the purified RSV particles may contain a range of about 50% to about 70%, such as about 50% to about 65%, about 50% to about 60%, or about 60% to about 65%, of the infectious titer of virus in the solution obtained by subjecting the host cell culture to TFF after subjecting the host cell culture to core bead chromatography.

The viral particles obtained by the purification methods described herein may retain infectivity following purification such that they can be used to induce a protective immune response when administered to a mammal.

Preparation of a fragile virus, such as hRSV, can be particularly challenging since virus particles can be damaged during binding to and elution from a chromatographic resin and by other forces such as shear. The processes disclosed herein may result in vaccine strain virus that is about 50 to about 200-fold more pure than the starting material with respect to Vero host cell proteins and DNA. Small-scale studies indicate that use of supernatant versus whole cell lysate as the starting material may result in purified preparations that are superior with respect to purity. Even still, it may be possible to optimize the purification procedure of the whole cell lysate to maximize yield and minimize the presence of contaminants.

In certain embodiments disclosed herein, the method for the purification of RSV particles from a host cell culture comprising RSV particles may result in removal of at least about 90%, such as at least about 95% or about at least about 99%, of the host cell (e.g., Vero cell) proteins and at least about 85%, such as at least about 90% or at least about 95%, of the residual host cell (e.g., Vero cell) DNA. Removal of these process stream contaminants may aid in the methods disclosed herein in order for improved scaled-up, modification, and optimization for preparation of clinical trial material. Thus, the data reported herein support the use of chromatography-based purification processes for preparation of RSV as well as other live-attenuated or wild type viral vaccines, suitable for testing in humans.

The RSV particles purified according to the present disclosure can be formulated according to known methods to prepare pharmaceutically useful compositions. The compositions of the disclosure can be formulated for administration to a mammalian subject, such as a human, using techniques known in the art. In particular, delivery systems may be formulated for intramuscular, intradermal, mucosal, subcutaneous, intravenous, injectable depot type devices or topical administration. When the delivery system is formulated as a solution or suspension, the delivery system may be in an acceptable carrier, such as an aqueous carrier. A variety of aqueous carriers may be used, e.g., water, buffered water, 0.8% saline, 0.3% glycine, hyaluronic acid and the like. These compositions may be sterilized by conventional, well-known sterilization techniques, or may be sterile filtered. The resulting aqueous solutions may be packaged for use as is, or lyophilized, the lyophilized preparation being combined with a sterile solution prior to administration.

The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

In certain embodiments disclosed herein, the pharmaceutical composition comprising the purified viral particles, including RSV particles, may comprise a buffer. The buffer may help to stabilize the virus during storage or may allow for cryopreservation of infectivity during multiple freeze thaw cycles. The selection of an appropriate buffer is important because RSV is fragile and loses its infectivity if stored in an incompatible buffer. Although extensive stability testing was not performed, some of the buffers that proved unsuccessful in stabilizing purified RSV during a single freeze/thaw cycle include 1× phosphate buffered saline (pH 7.4) with either 10% glycerol or 10% sorbitol or 20 mM citrate buffer (pH 7.2) with either 10% glycerol or 10% sorbitol.

In one embodiment, the purified RSV particles may be formulated into a buffer comprising sorbitol. In certain embodiments, the buffer comprises one or more of potassium glutamate, L-histidine, and sodium chloride. In another embodiment, the buffer comprises potassium glutamate, L-histidine, sodium chloride, and sorbitol. The buffer may have a pH ranging from about 7 to about 8, such as about 7.4. In one embodiments, the buffer comprises 50 mM potassium glutamate, 10 mM L-histidine, 160 mM sodium chloride, and 10% sorbitol, having a pH of about 7.4.

In certain exemplary embodiments, pharmaceutical compositions may be administered to mammalian subjects to induce an immune response in the mammalian subject. The intensity of such immune response may be modulated by dosage to range from a minimal response for diagnostic applications (e.g. skin testing for allergies) to a durable protective immune response (immunization) against challenge.

In order to enhance the immune response to the viral particle, such pharmaceutical compositions may optionally include adjuvants. Examples of adjuvants include aluminum salts (e.g. potassium aluminum sulfate, alum, aluminum phosphate, aluminum hydroxyphosphate, aluminum hydroxide), 3D-MPL, oil-in-water emulsions including but not limited to AS03, AF03, AF04, and QS21, and other adjuvants known to those in the art.

### EXAMPLES

The following examples are to be considered illustrative and not limiting on the scope of the disclosure described above.

### Example 1

### A. Methods

### Upstream Processing (USP)

Passage attenuated RSV was derived by forty-four serial passages on a naïve Vero cell line to evaluate the efficacy of a live attenuated vaccine approach (L. Zhang et al., to be published elsewhere). Viruses for purification, immunization and challenge, including live, passage-attenuated vaccine candidate virus, wild type MSA-1 strain and RSV long strain, were propagated on Vero cells grown to confluence in T-225 flasks. Vero cells were seeded at 1.8×10⁷ cells per flask and grown to confluence at 37° C., 5% CO₂ in DMEM supplemented with 10% Fetal Bovine Serum and 2 mM L-Glutamine. Flasks were then aspirated and cells infected with RSV at a multiplicity of infection (MOI) of 0.001 for 1 hour at 37° C., 5.0% CO₂ in 10 mL viral growth medium consisting of HyClone™ SFM4MegaVir™ (Thermo Scientific, Waltham, Mass.) supplemented with 2 mM L-Glutamine and 1× antibiotic/antimycotic (Thermo Scientific, Waltham, Mass.). After 1 hour, the flasks were aspirated, 40 mL fresh viral growth media was added to the virus-adsorbed cells, and the flasks were placed at 34° C., 5.0% CO₂. At 6 days post-infection (dpi), the RSV-containing media was harvested and processed as described below.

### Downstream Processing (DSP)

RSV-containing cell culture media was decanted from the T-225 flasks, which, as described above, had been infected with RSV (either LAV or wild type) 6 days earlier. The bulk harvest material was then clarified by centrifugation at 650×g for 5 minutes, and this clarified cell culture supernatant was considered the starting material. In the laboratory scale studies presented here, about 500 mL of virus containing material was processed at a time, whereas in the small scale discussed below in Example 2, about 30-60 mL of material was processed.

In order to reduce the amount of Vero DNA in the process stream, the solution was adjusted to 5 mM MgCl₂ and subsequently treated with Benzonase® (EMD/Merck, Darmstadt, Germany) endonuclease (90 U/mL, 5 hours, 25° C. with gentle agitation at 50 rpm).

Prior to chromatographic separation, the Benzonase®-treated sample was further clarified by depth filtration to remove cellular debris and/or aggregated material (0.65 μm SartoScale, SartoPure PP2, Sartorius Stedim, Goettingen, Germany). Briefly, the depth filtration manifold was assembled using ¼ inch tri-clover to hose-tail barb sterile flange fittings with the appropriate gaskets, tri-clover clamps and size 24 silicone MasterFlex® tubing (Cole-Parmer, Court Vernon Hills, Ill.). The entire manifold was sterilized as recommended by the manufacturer (autoclave 25 min. dry cycle at 121° C.), and the sample was processed at 90 mL/min by peristaltic pump (MasterFlex®, Cole Parmer) without preequilibration of the membrane.

Chromatographic separation of RSV virions from contaminating host cell proteins and DNA was performed on an AKTA Purifier (GE Healthcare Life Sciences, Piscataway, N.J.) located in a biosafety cabinet. A 45 mL Capto™ Core 700 column was packed into an empty XK26 column housing and then equilibrated into 1× phosphate buffered saline (PBS), as per the manufacturer's instructions (GE Healthcare Life Sciences). All subsequent chromatography steps were performed at 10 mL/min. The sample was applied to the column and virus containing material was collected as a single flowthrough (FT) fraction. Bound contaminants were removed from the column by cleaning in place (CIP) with 30% Isopropyl Alcohol prepared in 1M NaOH, as recommended by the manufacturer. FT and CIP fractions were collected manually while recording the ultraviolet absorbance at 280 nm.

Final concentration and formulation of the purified virus was accomplished by ultrafiltration/diafiltration (UF/DF) using a hollow fiber tangential flow filtration (TFF) apparatus (Kros-Flo Research II, Spectrum Laboratories, Rancho Dominguez, Calif.). An 85 cm², 500 kDa molecular weight cutoff (MWCO) polysulfone hollow fiber TFF module was used under low flow rate recirculation conditions to minimize shear force (130 mL/min). Transmembrane pressure (TMP) was kept below 4 psi throughout diafiltration to minimize formation of a gel layer, which thereby could impede fluid flux.

The virus was formulated into a buffer that allows for cryopreservation of infectivity during multiple freeze/thaw cycles. The buffer comprised 50 mM potassium glutamate, 10 mM L-histidine, 160 mM NaCl, and 10% sorbitol and had a pH of 7.4.

### Characterization of Purification Process Retains Infectivity

Potency of virus containing fractions was interrogated by titration on naïve Vero cells, which were originally obtained from the American Type Culture Collection (ATCC, Manassas, Va.). Cells were maintained in DMEM (Life Technologies) supplemented with 10% FBS, 2 nM L-glutamine and 1× antibiotic/antimycotic mixture at 37° C. and 5% CO₂. Infectivity was assessed by titration of purification process retains by plaque assay, as has been described previously (Murphy, B. R., et al., Vaccine, 1990; 8:497-502). Plaques were visualized by immunostaining with Horseradish Peroxide conjugated goat anti-RSV antibody (Abcam AB20686). Titers were determined by counting stained plaques and are expressed herein as plaque forming units (PFU) per mL.

Purity (SDS-PAGE, Western Blotting, Vero HCP ELISA, and Vero DNA qPCR)

RSV containing samples were resolved by 4-12% SDS-PAGE (NuPAGE®, Bis-Tris, Life Technologies) after heating of the samples for 5 minutes at 95° C. in Laemmli SDS sample loading buffer containing β-mercaptoethanol (Boston BioProducts, Ashland, Mass.). The poylacrylamide gels were either stained with SimplyBlue™ SafeStain (Life Technologies) or transferred to a nitrocellulose membrane using a dry protein transfer on the iBlot transfer apparatus (Life Technologies). The membranes were probed using the following mouse monoclonal antibodies (mAbs): anti-RSV F (Sanofi Pasteur); commercially-available mouse anti-RSV M2-1 (RSV5H5) (Abcam, Cambridge, UK); and anti-RSV G Glycoprotein (RSV133) (Abcam, Cambridge, UK). Membranes were subsequently incubated with an alkaline phosphatase-labeled anti-mouse IgG secondary antibody (Southern Biotech, Birmingham, Ala.), and proteins were visualized using the SIGMAFAST™ BCIP®/NBT (Sigma-Aldrich, St. Louis, Mo.) chromogenic reagent.

A commercially available ELISA for Vero host cell proteins (HCP) (Cygnus Technologies, Southport, N.C.) was utilized to determine the purity of process retains as well as of purified virus preparations. The ELISA was performed as per the manufacturer's instructions, except that the following diluent was used in sample preparation for the HCP ELISA: 50 mM Tris, 0.1 M NaCl, 8 mg/mL bovine serum albumin, pH 7.0. A quantitative PCR (qPCR) assay was used to measure concentration of contaminating Vero DNA.

### Other Analysis (Electron Microscopy and Mass Spectrometry)

For visualization of viral particles and filaments, purification process retains were analyzed by transmission electron microscopy (TEM). Microscopy conditions were as follows: positive stain with 1.5% uranyl acetate, direct magnification between 12,000 and 100,000× on a JEOL® JEM-1010 general purpose TEM with digital image acquisition capability.

To confirm the identity of protein bands on SDS gel, each band was cut and in gel digestion was performed with trypsin on an Intavis Robot. The tryptic peptides were then analyzed by Nano LC-MS/MS on Thermo Velos Orbitrap. The protein identity of major bands was identified by searching an RSV database.

### Animal Procedures and Analysis

Young adult female cotton rats (4 to 6 weeks of age) were housed at Sigmovir Biosystems, Inc. (Maryland, N.J.). Following a pre-bleed, cotton rats were randomly separated into 3 groups of 6 animals and immunized intramuscularly (IM) with 0.1 mL PBS containing 10E+4 PFU of RSV. On day 28 post-immunization, the animals were bled and then challenged intranasally (IN) with 10E+5 PFU of RSV long strain. Four days post challenge, the animals were euthanized via CO₂ intoxication, and their lungs and noses were removed and homogenized in Leibovitz's L-15 Medium (Thermo Scientific, Waltham, Mass.) supplemented with a sucrose-phosphate-glutamate freezing buffer comprising 74.62 g/l sucrose, 0.517 g/l KH₂PO₄, 1.25 g/l K₂HPO_{4.3}, and 0.829 g/l sodium glutamate, frozen on dry ice and shipped for analysis.

Serum samples were analyzed for RSV-specific neutralizing antibody titers as follows: serum was heat inactivated for 30 minutes at 56° C. A four-fold serial dilution series of the inactivated serum was made in Eagle's minimum essential media (EMEM) with Earle's BSS (Lonza, Basel Switzerland). RSV viral stocks were diluted to 2×10⁴ PFU/mL, combined 1:1 with the serum dilutions, and incubated for 1 hour at 30° C. The virus-serum mixture was then added to 24 well plates containing confluent Hep2 cell monolayers at 50 μL per well and incubated for 1 hour at 37° C., 5% CO₂. The inoculum was then overlaid with 1 mL per well of 0.75% methyl cellulose in EMEM supplemented with 10 mL fetal bovine serum, 2 mM L-glutamine, 50 μg/ml Gentamicin and 2.5 μg/mL Fungizone (all from Lonza, Basel Switzerland). Following a 4 day incubation at 37° C., 5% CO₂, overlay was removed and the monolayers fixed and stained with Crystal Violet in 5% glutaric dialdehyde for 3 hours at 25° C. Plates were washed 3 times with water, air-dried, and the plaques counted using a dissecting microscope. The neutralizing antibody titers were determined at the 60% reduction endpoint of mock neutralized virus controls using the formula:

60% plaque reduction titer=(C/V×0.4−Low)/(High−Low)×(HSD−LSD)+LSD

where C/V equals the average of RSV plaques in mock neutralized virus control wells. Low and High are the average number of RSV plaques in the two dilutions that bracket the C/V×0.4 value for a serum sample, and the HSD and LSD are the Higher and Lower Serum Dilutions. RSV titers in nasal and lung homogenates were determined essentially as per the viral stock titration protocol, except that following the 1 hour viral adsorption step, the wells were aspirated before the addition of overlay to minimize inhibition and the titers were reported as PFU per gram of tissue (PFU/g). Comparisons of neutralizing antibody concentrations and viral titers between groups of cotton rats were performed by two-tailed Mann Whitney t-test.

### B. Results

Infectious Virus Yield from Core Bead Chromatography Purification Scheme

hRSV LAV and wild type strain MSA-1 were purified by a four step purification process as discussed above that included DNA reduction, clarification, core bead chromatography and hollow fiber TFF unit operations. Recovery of infectious virus at each step of the purification procedure was confirmed by plaque assay. Surprisingly, recovery of infectious virus at the end of the chromatography/TFF purification process was about 50-60% overall. It was also surprising to discover that there was virtually no reduction in titer through the chromatography step, irrespective of which virus strain was being purified. See Table 1, columns 5 and 6, below. By comparison, previous attempts to purify RSV by an ion exchange chromatography-based purification scheme resulted in recovery of only about 1% of infectious virus (Downing, L. A., et al., J Virol Methods, 1992; 38:215-28). The starting material was concentrated about 10-fold by volume, which corresponded to a 1 log increase in titer. See Table 1, columns 3 and 4, below. Virtually all losses of infectious virus occurred during the final concentration and buffer exchange step (TFF).

Table 1 below shows live-attenuated virus (LAV) and wild type RSV infectious virus yield from core bead/TFF purification procedure. Core FT refers to the flow through from the chromatography step. Final refers to the virus-containing solution after the TFF step. The data in Table 1 are from plaque assays performed on unfrozen purification retains. The chromatography/TFF-based purification process results in a recovery of about 50-60% of the infectious virus and concentration of titers about 10-fold.

TABLE 1
LAV and Wild Type RSV Infectious Virus Yield

| RSV | Retain | Volume | Titer | Total | |
|---|---|---|---|---|---|
| Strain | (mL) | (PFU/mL) | (PFU) | Virus | Recovery^{a} |
| LAV | Start | 500 | 3.4E+7 | 1.7E+10 | 100% |
| | Benzonase | 500 | 3.2E+7 | 1.6E+10 | 94% |
| | 0.65 μM | 490 | 3.5E+7 | 1.7E+10 | 100% |
| | Core FT | 500 | 3.5E+7 | 1.75E+10 | 102% |
| | Final | 38 | 2.15E+8 | 8.2E+9 | 48% |
| WT | Start | 500 | 8.2E+5 | 4.1E+8 | 100% |
| | Benzonase | 500 | 8.0E+5 | 4.0E+8 | 97.5% |
| | 0.65 μM | 490 | 7.0E+5 | 3.4E+8 | 83% |
| | Core FT | 500 | 8.2E+5 | 4.1E+8 | 100% |
| | Final | 32.5 | 7.1E+6 | 2.3E+8 | 56% |

^{a}Recovery is presented as a % of the total titer at the start of the purification.

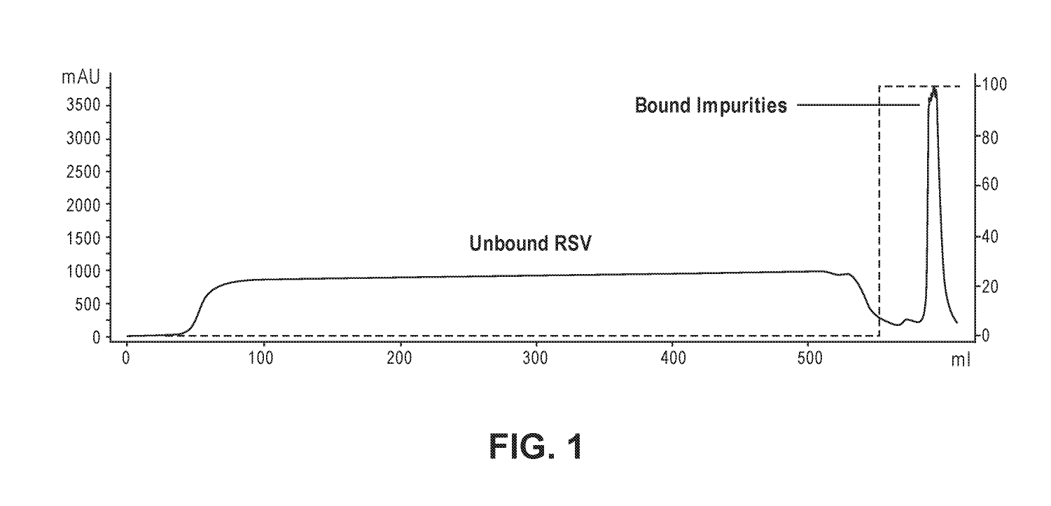

As shown in FIG. 1, the chromatographic profile appeared as expected with the virus containing material flowing through the column and lower molecular weight contaminants binding the resin and subsequently being stripped from the column by CIP. In FIG. 1, the solid line represents absorbance at 280 nm, and the dotted line represents the concentration of Buffer B, a Cleaning-In-Place (CIP) solution of 0.5 M NaOH in 30% isopropyl alcohol, which followed the sample flowthrough phase to remove bound impurities, including residual Vero host cell proteins and DNA.

### Characterization of Purity and Integrity of Purified Virions

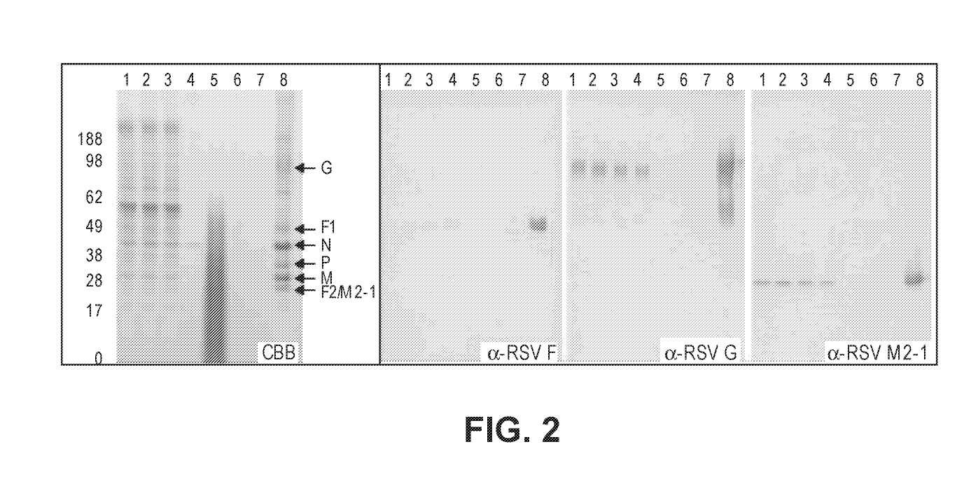

The removal of major process stream contaminants (such as Vero HCP and DNA) was confirmed using a variety of analytical techniques. SDS-PAGE and Western blotting reveal that throughout the initial purification steps, the ratio of viral proteins to Vero host cell proteins remains the same. As shown in FIG. 2, a comparison of unpurified (FIG. 2, lane 1), Benzonase®-treated (FIG. 2, lane 2), and 0.65 μm depth-filtered (FIG. 2, lane 3) purification fractions demonstrates the relative stability in protein level. Post-chromatography, although the intensity of the viral protein bands on the Western blot remains the same, the overall protein in the Coomassie-stained gel drops dramatically. See FIG. 2, lane 3 in comparison to FIG. 2, lane 4. These data are in accordance with the infectivity data presented in Table 1 above, as well as the Vero HCP ELISA data presented in Table 2, below.

TABLE 2
Chromatography-Based Purification Process Results in About 50-200-Fold
Purification Factor with Respect to Vero Host Cell Proteins (HCP)

| RSV | Retain | Vero HCP | | | |
|---|---|---|---|---|---|
| Strain | (μg/mL) | (total mg) | (PFU/μg) | Factor | Purification |
| LAV | Start | 48.2 | 24.1 | 7.0E+11 | 1× |
| | Benzonase | 42.4 | 21.2 | 7.6E+11 | 1× |
| | 0.65 μM | 42.2 | 20.7 | 8.4E+11 | 1× |
| | Core FT | 0.6 | 0.3 | 6.1E+13 | 87× |
| | Final | 5.9 | 0.2 | 3.6E+13 | 51× |
| WT | Start | 19.5 | 9.7 | 4.2E+10 | 1× |
| | Benzonase | 17.9 | 8.9 | 4.5E+10 | 1× |
| | 0.65 μM | 17.3 | 8.5 | 4.0E+10 | 1× |
| | Core FT | 0.1 | 0.04 | 1.2E+13 | 285× |
| | Final | 1.00 | 0.03 | 7.1E+12 | 169× |

The results presented in Table 2 highlight the increase in purity with respect to Vero HCP that occurs during the column chromatography step. About 99% of the Vero HCP binds to the core bead resin, and about 100% of the infectious virus flows through the resin. The intensity of RSV proteins by SDS-PAGE and by Western blot are visibly increased post TFF. See FIG. 2, comparing lane 3 to lane 8. This corresponds with a 10-fold decrease in volume and an increase in titer of 1 log, as expected since infectivity of the virus is preserved throughout the purification process. See Table 1, above. The identity of the major protein bands in the purified virus preparation was confirmed by Nano LC-MS/MS (data not shown), and the bands are labeled as identified. See FIG. 2, lane 8, showing the TFF retentate of purified RSV.

An even higher degree of purity was obtained as concerns residual Vero DNA. A 50-fold reduction in Vero DNA levels post-Benzonase® endonuclease treatment and then an additional 4-fold reduction after depth filtration and column chromatography resulted in an approximate 100-200-fold reduction in the amount of Vero DNA as compared with the starting material. See Table 3, below. For the LAV strain virus, there were as many as 1.1×10⁸ PFU of LAV per 10 ng of Vero DNA in the fully purified material.

TABLE 3
Chromatography-Based Purification Process Results in Purification
Factor 2 Orders of Magnitude with Respect to Residual Vero DNA

| | | Residual Vero DNA | | | |
|---|---|---|---|---|---|
| RSV | Retain | | (PFU/10 | | |
| Strain | (ng/mL) | (total μg) | ng) | Factor | Purification |
| LAV | Start | 312 | 156 | 1.1E+6 | 1× |
| | Benzonase | 5.8 | 2.9 | 5.5E+7 | 50× |
| | Core FT | 1.7 | 0.8 | 2.1E+8 | 190× |
| | Final | 19.7 | 0.75 | 1.1E+8 | 100× |
| WT | Start | 435 | 21.75 | 1.9E+4 | 1× |
| | Final | 18.2 | 0.6 | 3.9E+6 | 205× |

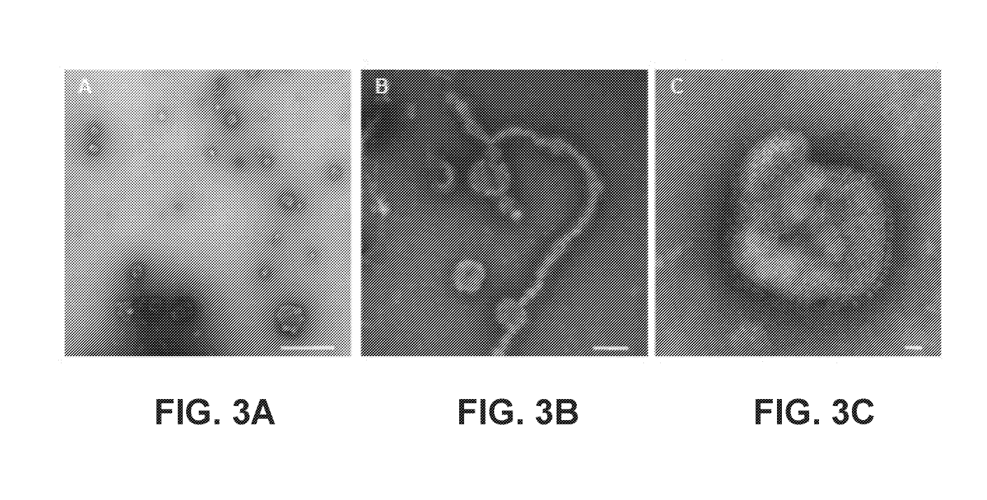

Finally, as a means to assess the integrity of the virus, particles were visualized by TEM at all stages of the purification process. The electron micrographs displayed in FIG. 3A-C are representative of what was seen across all infectious virus containing purification fractions. As expected of a pleiomorphic virus, morphology of virions was a mixture of spherical, filamentous, and intermediate forms. These virion forms were represented in all stages of the purification process, though the micrographs presented herein and shown in FIGS. 3A, 3B, and 3C were obtained from the core bead flowthrough fraction. Therefore the virions presented herein represent partially-purified and not fully concentrated material. At all stages of the purification single viral particles and filaments were observed. At higher magnifications glycoprotein spikes can be seen at the surface of the viral envelope.

In Vivo Immunogenicity and Efficacy of Crude Vs. Purified LAV

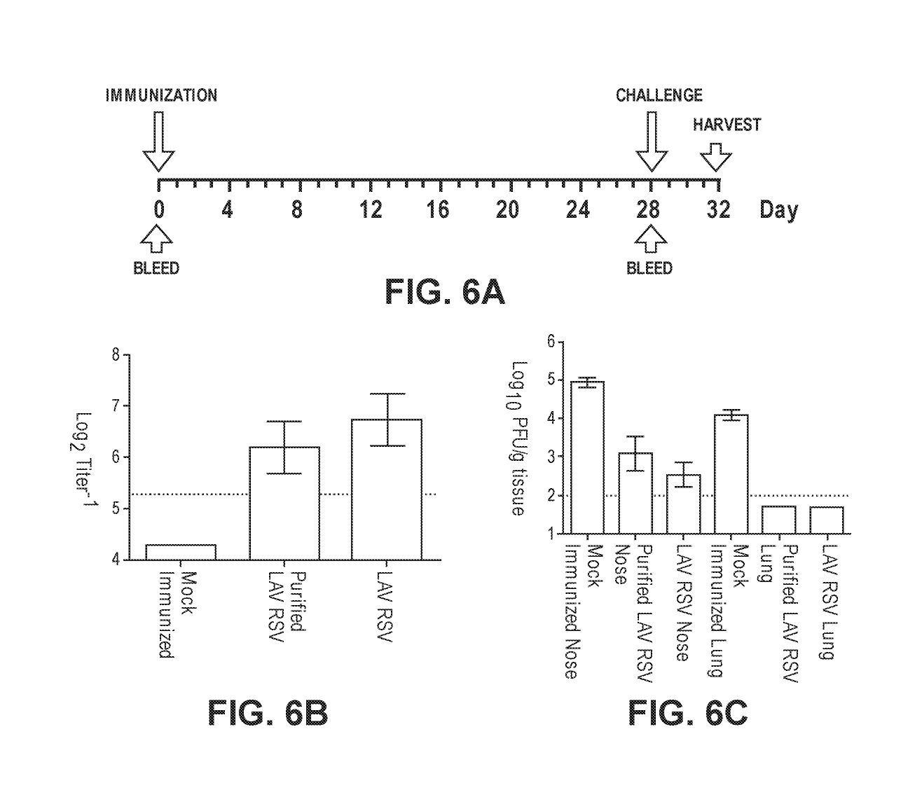

Groups of 6 cotton rats received intramuscular injections of 10E+4 PFU of purified or unpurified LAV in 0.1 mL PBS or were mock immunized. FIG. 6A illustrates a timeline of the study schedule. Blood was collected 28 days following immunization, and the RSV neutralizing antibody titers in the serum were determined. FIG. 6B graphically illustrates the neutralizing antibody titers in the serum collected, wherein the dotted line represents the limit of detection. Animals immunized with purified and unpurified LAV exhibited similar neutralizing antibody titers (medians 6.25 and 6.98 log 2, P=0.3874), while all mock immunized animals had titers below the limit of detection, as did all day 0 pre-bleed samples (data not shown).

To assess the protective efficacy of crude versus purified LAV, the animals were challenged intranasally with 10E+5 PFU of RSV long strain on day 28 post immunization, and 4 days later their RSV lung and nasal titers were determined. FIG. 6C graphically illustrates the lung and nasal RSV titers. The dotted line represents the limit of detection. Both immunized groups were completely protected from lower respiratory tract infection (LRI) with lung titers falling below the limit of detection, while the mock immunized group exhibited a median 4.15 log 10 PFU/g. In the upper respiratory track (URT), animals immunized with purified and unpurified LAV exhibited similarly low RSV titers (medians 2.95 and 2.15 log 10 PFU/g, P=0.2316), that were at least 100 fold lower than those of the mock immunized animals (P<0.005).

Purified and unpurified LAV were similarly immunogenic in vivo and showed comparable levels of protective efficacy against challenge in the upper and lower respiratory tract. While the median neutralizing antibody titer in the purified LAV immunized group was marginally lower and the URT RSV titer was marginally higher than that in the unpurified LAV immunized group, this trend did not approach significance. That purified LAV did not show a significant loss of immunogenicity despite the large reduction in Vero HCP and DNA levels indicates that this construct is highly immunogenic. The immunogenicity and protective efficacy of LAV can be further increased by larger and multiple doses (data not shown).

### Example 2—Small-Scale Lysate Preparation and Purification Study

An additional small-scale study was performed to determine if a whole cell lysate could be appropriate starting material for purification. The methods used were a scaled down version to what is described above except that in addition to clarified cell culture supernatant, whole cell lysate was utilized as the starting material for purification. To test conditions for lysate preparation, T-225 flasks of infected cells were harvested by scraping the cells from the flask. Cell disruption was accomplished either by sonication using a Branson Sonifier Cell Disruptor equipped with a microtip, 60 seconds on ice, 50% duty cycle, output level 6 (Branson Ultrasonics Corp., Danbury Conn.), or by microfluidization using a M-110Y high pressure pneumatic, 1 vs. 3 passes on ice at 2,500 pounds per square inch (psi) or 1 vs. 3 passes on ice at 5,000 psi (Microfluidics Corp., Westwood, Mass.).

At the small scale, samples were then treated with Benzonase® endonuclease as before and filtered through a 0.8 μm flat sheet polyethersulfone (PES, Supor®) membrane syringe filter (Pall Corp., Port Washington, N.Y.). Samples were loaded onto a 5 mL Capto™ Core 700 column poured in an XK16 column housing at 5 mL/min. TFF was not performed at the small scale.

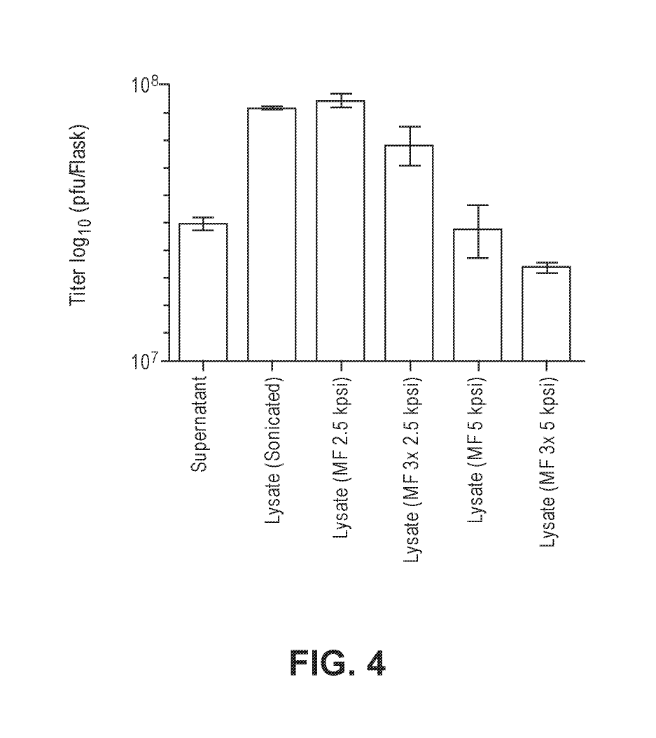

To ascertain whether higher titer material could be prepared from a whole cell lysate, sonication and microfluidization were explored as means for mechanical cell disruption. It had been previously noted that sonication could release more infectious virus from the cells than that released into the supernatant post infection. This was also observed in the current study, where sonication resulted in about a 2-fold increase of infectious virus per flask after centrifugation and clarification. FIG. 4 is a graph demonstrating that sonication and low-pressure microfluidization resulted in higher titers as compared to the amount of infectious virus in the clarified cell culture supernatant. Low pressure microfluidization (2.5 kpsi) produced a similar result to sonication, although at higher pressure the benefit of microfluidization was not realized. See FIG. 4.

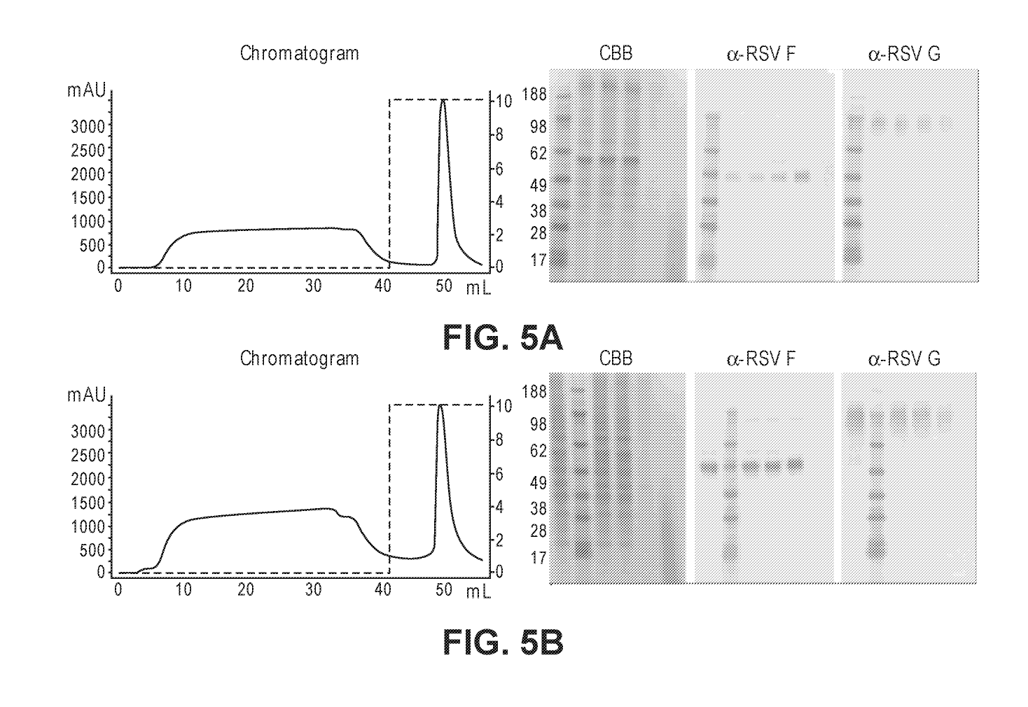

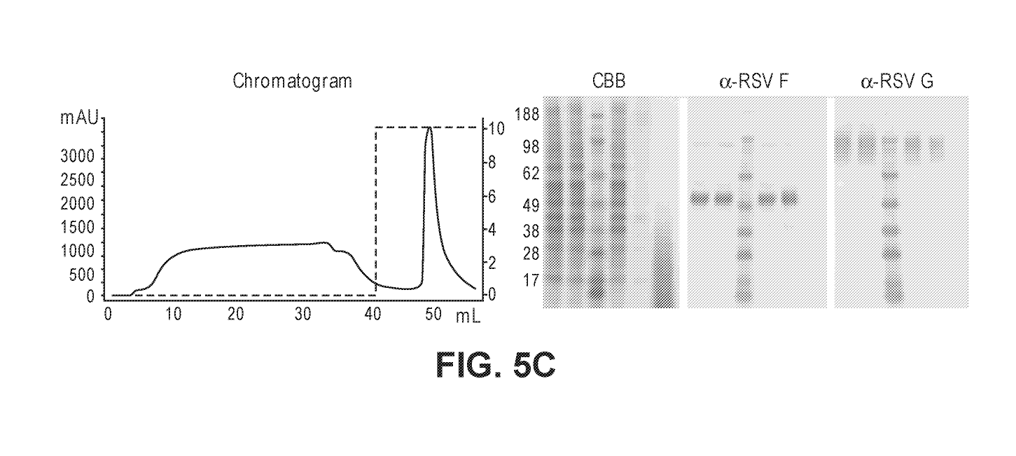

A small-scale comparison of the initial purification steps was undertaken to assess infectivity and purity using either cell culture supernatant (as was used in the laboratory scale experiments described above in Example 1) or lysate prepared either by sonication or by microfluidization. Initial comparison of chromatograms as well as SDS-PAGE and Western Blot indicate that although the chromatographic elution profiles remained the same, there were differences between the treatments. FIGS. 5A-5C show the comparison of initial purification steps using supernatant (FIG. 5A), whole cell lysate prepared by sonication (FIG. 5B), and whole cell lysate prepared by microfluidization (FIG. 5C) as the bulk harvest material. SDS-PAGE of fractions from the lysate preparations in FIGS. 5B and 5C appeared to contain much more background proteins as compared with the supernatant (FIG. 5A), although they also contained more viral proteins as indicated by the Western blots.

Infectivity of the fractions was followed by plaque assay, as done previously. See Table 4A, below. As shown in Tables 4A-C, two-fold infectivity of lysate, prepared either by sonication or by low-pressure microfluidization, vs. cell culture supernatant did not offset the decrease in purity due to process stream contamination by host cell proteins and DNA. See Tables 4B and 4C. Purification factor, which may be defined as fold purification from contaminants, was comparable irrespective of the nature of the starting material.

TABLE 4A
Infectivity

| | Retain | Volume | Titer | Total | |
|---|---|---|---|---|---|
| Harvest | (mL) | (PFU/mL) | (PFU) | Virus (%) | Recovery^{a} |
| Supernatant | Start | 37 | 1.9E+7 | 7.2E+8 | 100% |
| | Benzonase | 37 | 1.8E+7 | 6.5E+8 | 91% |
| | 0.8 μm | 36 | 1.3E+7 | 4.7E+8 | 65% |
| | Core FT | 40 | 1.2E+7 | 5.8E+8 | 80%^{b} |
| Sonicated | Start | 37 | 4.8E+7 | 1.8E+9 | 100% |
| | Benzonase | 37 | 4.2E+7 | 1.5E+9 | 88% |
| | 0.8 μm | 34 | 3.9E+7 | 1.3E+9 | 75% |
| | Core FT | 40 | 2.3E+7 | 1.0E+9 | 60%^{b} |
| Microfluidized | Start | 37 | 3.8E+7 | 1.4E+9 | 100% |
| | Benzonase | 37 | 2.8E+7 | 1.0E+9 | 74% |
| | 0.8 μm | 34 | 3.4E+7 | 1.2E+9 | 83% |
| | Core FT | 40 | 2.2E+7 | 1.0E+9 | 74%^{b} |

^{a}Recovery is presented as a % of the total titer at the start of the purification
^{b}Adjusted recovery, only 30 mL of filtered material was run on Capto ™ Core 700 column

As could be expected from the intensity of the viral protein bands on the Western blot, there were about 2-fold more PFU per fraction in the preparations where the lysate was used. Recoveries after initial purification steps were slightly lower (about 60-80%) than at the laboratory scale, although this may be explained by differences in preparation (such as filtration, etc.) that were implemented at this scale. Fold-purification from contaminants after the initial purification steps were comparable, regardless of the starting material that was used, but the purity of the material was decreased when lysate was used versus cell culture supernatant. See Tables 4B and 4C, below.

TABLE 4B
Vero HCP

| | Retain | Vero HCP | | | |
|---|---|---|---|---|---|
| Harvest | (μg/mL) | (total mg) | (PFU/μg) | Factor | Purification |
| Supernatant | Start | 48.4 | 1.8 | 3.9E+11 | 1× |
| | Core FT | 0.56 | 0.02 | 2.1E+13 | 54× |
| Sonicated | Start | 284 | 10.5 | 1.7E+11 | 1× |
| | Core FT | 5.4 | 0.2 | 4.2E+12 | 25× |
| Microfluidized | Start | 312 | 11.6 | 1.2E+11 | 1× |
| | Core FT | 2.85 | 0.1 | 7.7E+12 | 64× |

TABLE 4C
Residual Vero DNA

| | | Residual Vero HCP | | | |
|---|---|---|---|---|---|
| | Retain | | (PFU/ | | |
| Harvest | (ng/mL) | (total μg) | 10 ng) | Factor | Purification |
| Supernatant | Start | 1260 | 46.6 | 1.5E+5 | 1× |
| | Core FT | 10 | 0.4 | 1.2E+7 | 80× |
| Sonicated | Start | 8250 | 305 | 5.8E+4 | 1× |
| | Core FT | 31 | 1.2 | 7.4E+6 | 127× |
| Microfluidized | Start | 11400 | 422 | 3.3E+4 | 1× |
| | Core FT | 34 | 1.3 | 6.5E+6 | 196× |

It is also noted that, as used in this disclosure and the appended claims, the singular forms “a”, “an”, and “the” include plural referents unless the context clearly dictates otherwise. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination). Ranges may be expressed herein as from about one particular value, and/or to about another particular value. When such a range is expressed, another aspect includes from the one particular value and/or to the other particular value. Similarly, when values are expressed as approximations, by use of the antecedent about, it will be understood that the particular value forms another aspect. It will be further understood that the endpoints of each of the ranges are significant both in relation to the other endpoint, and independently of the other endpoint. All references cited in this disclosure are hereby incorporated herein in their entirety.

1. A method for the purification of Respiratory Syncytial Virus (RSV) particles from a host cell culture comprising RSV particles, the method comprising the steps of:
a) treating the host cell culture with an endonuclease;
b) filtering the material from step (a) to remove cellular debris and/or aggregated material;
c) applying the material obtained from step (b) to a core bead chromatography resin such that the RSV particles flow through the resin; and
d) recovering the purified RSV particles.

2. The method according to claim 1, further comprising subjecting the RSV particles recovered in step (d) to tangential flow filtration.

3. The method according to claim 1, wherein the purified RSV particles contain greater than about 1×10⁷ plaque forming units (PFU)/mL.

4. The method according to claim 1, wherein the purified RSV particles contain greater than about 1×10⁸ PFU/mL.

5. The method according to claim 1, wherein the endonuclease is from Serratia marcescens and comprises two subunits, each of which has a molecular weight of about 30 kD, and degrades double stranded and single stranded DNA and double stranded and single stranded RNA.

6. The method according to claim 1, wherein the purified RSV particles contain less than 10 ng host cell DNA per 1×10⁷ PFU.

7. The method according to claim 1, wherein greater than about 99% of the host cell protein and greater than about 95% of the host cell DNA is removed in the recovered purified RSV particles.

8. The method according to claim 1, wherein about 50-60% of the infectious RSV titer from the host cell culture remains following the tangential flow filtration step.

9. The method according to claim 1, wherein the tangential flow filtration is a hollow fiber system.

10. The method according to claim 1, wherein about 100% of the infectious RSV titer from the host cell culture remains following the core bead chromatography step.

11. A pharmaceutical composition comprising Respiratory Syncytial Virus (RSV) produced in a cell culture, said RSV isolated by the method comprising the steps of:
a) treating the host cell culture with an endonuclease;
b) filtering the material from step (a) to remove cellular debris and/or aggregated material;
c) applying the material obtained from step (b) to a core bead chromatography resin such that the RSV particles flow through the resin;
d) recovering the purified RSV particles; and
e) suspending the purified RSV particles in a pharmaceutically acceptable carrier.

12. The pharmaceutical composition of claim 11, wherein the method further comprises the step of subjecting the RSV particles recovered in step (d) to tangential flow filtration.

13. The pharmaceutical composition of claim 11, wherein the pharmaceutical composition comprises a buffer comprising sorbitol.

14. The pharmaceutical composition of claim 11, wherein the purified RSV particles contain greater than about 1×10⁷ plaque forming units (PFU)/mL.

15. The pharmaceutical composition of claim 11, wherein the purified RSV particles contain greater than about 1×10⁸ PFU/mL.

16. The pharmaceutical composition of claim 11, wherein the endonuclease is from Serratia marcescens and comprises two subunits, each of which has a molecular weight of about 30 kD, and degrades double stranded and single stranded DNA and double stranded and single stranded RNA.

17. The pharmaceutical composition of claim 11, wherein the purified RSV particles contain less than 10 ng host cell DNA per 1×10⁷ PFU.

18. The pharmaceutical composition of claim 11, wherein greater than about 99% of the host cell protein and greater than about 95% of the host cell DNA is removed in the recovered purified RSV particles.

19. The pharmaceutical composition of claim 11, wherein about 100% of the infectious RSV titer from the host cell culture remains following the core bead chromatography step.

20. The pharmaceutical composition of claim 11, wherein the tangential flow filtration is a hollow fiber system.

21. The pharmaceutical composition of claim 11, wherein about 50-60% of the infectious RSV titer from the host cell culture remains following the tangential flow filtration step.
